(19) United States
(12) Patent Application Publication
(10) Pub. No.: US 2022/0108781 A1
(43) Pub. Date: Apr. 7, 2022

(54) COMPUTERIZED ROUTE BUILDING SYSTEM AND METHOD

(71) Applicant: PAVÉ ROUTES, LLC, Canton, OH (US)

(72) Inventor: Sebastian S. Hendricks, Canton, OH (US)

(21) Appl. No.: 17/496,118

(22) Filed: Oct. 7, 2021

Related U.S. Application Data
(60) Provisional application No. 63/088,590, filed on Oct. 7, 2020.

(51) Int. Cl. G16H 20/30; H04W 4/024; G01C 21/34; G01C 21/36

(52) U.S. Cl. CPC G16H 20/30; H04W 4/024; H04W 4/90; G01C 21/3635; G01C 21/3484

(57) ABSTRACT

A system for creating a customized route to travel including a mobile device having a human interface for inputting user-determined criteria, memory on which processor executable instructions are stored, a processor in communication with the human interface and the memory, and a mobile display for displaying the customized route to travel. A system server is configured for analyzing user-determined criteria and for generating the customized route to travel, the system server in communication with the mobile device. The customized route to travel is created based on user-determined criteria, which is transmitted to the mobile device. A display of the customized route to travel is generated on the mobile display of the mobile device, the display including a visual map of the customized route to travel and real-time information regarding the exercise plan including guidance and user feedback.

### CROSS-REFERENCE TO RELATED APPLICATIONS

This application claims the benefit of U.S. Provisional Application No. 63/088,590, filed on Oct. 7, 2020. The entire disclosure of the above application is hereby incorporated herein by reference.

### FIELD

The present technology relates to methods, systems, and storage media for creating a customized exercise plan.

### INTRODUCTION

This section provides background information related to the present disclosure which is not necessarily prior art.

People travel by a wide variety of means. Examples of such means include running, walking, hiking, biking, driving, and the like. Sometimes people travel for necessity, and sometimes for physical fitness, and other times for recreation. Indeed, people are known to travel for a variety of additional reasons.

There are numerous paths for people to travel. Such paths include roads, sidewalks, hiking trails, bike trails, and so on. Many of such paths are captured in maps. Maps showing the streets/highways located in a given area are common. Maps showing paths like bike trails and hiking trails also exist in a variety of forms. Many people are familiar with paper maps, which have been available for many years. Electronic maps have also emerged as a viable source for viewing several types of paths in recent decades.

People who travel often consult such maps. Sometimes, people consult maps before traveling in order to plan their travel routes. Other times, people consult maps after traveling in order to see where they have been and how far they have traveled. People may gain valuable information about their travel by consulting maps.

In addition to maps, people often consult other sources of information while making travel plans. For example, people may seek information related to weather, road construction, land-marks, and various other items of information. To gather this other information, people may be required to consult several sources.

Known types of maps, in both their paper and electronic forms, can be difficult to use for people who want to use customized routes for their travel, exercise or recreation. Such maps are often predetermined according to the platform being used, and may only suggest preset routes based on a limited number of factors not necessarily tailored to the needs or desires of the user.

There is a continuing need for a route building system and method that is easily accessible and provides desired information regarding the use of the generated routes. Desirably, the route building system and method permits for a superior customization of generated routes compared to known systems.

### SUMMARY

In concordance with the instant disclosure, a route building system and method that is easily accessible and provides desired information regarding the use of the generated routes, and which permits for a superior customization of generated routes compared to known systems, has been surprisingly discovered.

In one embodiment, the present disclosure relates to a method for creating a customized route to travel. The method may include providing a system for creating a customized route to travel, the system including a mobile device having a human interface for inputting user-determined criteria, a memory on which processor executable instructions may be stored, a processor in communication with the human interface and the memory, and a mobile display for displaying the customized route to travel compiled by the system server. The system server may be configured for analyzing the user-determined criteria and for generating the customized route to travel. A system server may be in communication with the mobile device through a wide area network. The method may include permitting the user, by human interface of the mobile device, to select the user-determined criteria including a duration of the exercise, a pattern of the exercise, a level of difficulty of the exercise, an inclusion of obstacles in the exercise, a general location of the exercise, and a presence of visual stimuli during exercise. The method may include transmitting the user-determined criteria from the mobile device to the system server. The method may include creating, by the system server, the customized route to travel based on user-determined criteria. The method may include transmitting the customized route to travel from the system server to the mobile device through the wide area network. The method may include generating a display of the customized route to travel on the mobile display of the mobile device, the display of the customized route to travel including a visual map of a route associated with the customized route to travel. The method may include providing, by the mobile device, real-time information to the user regarding the exercise plan including guidance and user feedback.

In another embodiment, the present disclosure relates to a system for creating a customized route to travel. The system may include one or more hardware processors configured by machine-readable instructions to create a customized route to travel. The machine-readable instructions may be configured to provide a system for creating a customized route to travel. The system may include a mobile device having a human interface for inputting user-determined criteria, a memory on which processor executable instructions be stored, a processor in communication with the human interface and the memory, and a mobile display for displaying the customized route to travel compiled by the system server. A system server may be configured for analyzing the user-determined criteria and for generating the customized route to travel, the system server in communication with the mobile device through a wide area network. The machine-readable instructions may be configured to permit the user, by human interface of the mobile device, to select the user-determined criteria including a duration of the exercise, a pattern of the exercise, a level of difficulty of the exercise, an inclusion of obstacles in the exercise, a general location of the exercise, and a presence of visual stimuli during exercise. The machine-readable instructions may be configured to transmit the user-determined criteria from the mobile device to the system server. The machine-readable instructions may be configured to create, by the system server, the customized route to travel based on user-determined criteria. The machine-readable instructions may be configured to transmit the customized route to travel from the system server to the mobile device through the wide area network. The machine-readable instructions may be configured to generate a display of the customized route to travel on the mobile display of the mobile device, the display of the customized route to travel including a visual map of a route associated with the customized route to travel. The machine-readable instructions may be configured to provide, by the mobile device, real-time information to the user regarding the exercise plan including guidance and user feedback.

In yet another embodiment, the present disclosure relates to a computer-readable storage medium for creating a customized route to travel. In some embodiments, the computer-readable storage medium may include instructions being executable by one or more processors for providing a system for creating a customized route to travel. The system may include a mobile device having a human interface for inputting user-determined criteria, a memory on which processor executable instructions may be stored, a processor in communication with the human interface and the memory, and a mobile display for displaying the customized route to travel compiled by the system server. A system server may be configured for analyzing the user-determined criteria and for generating the customized route to travel. The system server may be in communication with the mobile device through a wide area network. In some embodiments, the computer-readable storage medium may include instructions being executable by one or more processors to permit the user, by human interface of the mobile device, to select the user-determined criteria including a duration of the exercise, a pattern of the exercise, a level of difficulty of the exercise, an inclusion of obstacles in the exercise, a general location of the exercise, and a presence of visual stimuli during exercise. In some embodiments, the computer-readable storage medium may include instructions being executable by one or more processors to transmit the user-determined criteria from the mobile device to the system server. In some embodiments, the computer-readable storage medium may include instructions being executable by one or more processors to create, by the system server, the customized route to travel based on user-determined criteria.

In some embodiments, the computer-readable storage medium may include instructions being executable by one or more processors to transmit the customized route to travel from the system server to the mobile device through the wide area network. In some embodiments, the computer-readable storage medium may include instructions being executable by one or more processors to generate a display of the customized route to travel on the mobile display of the mobile device, the display of the customized route to travel including a visual map of a route associated with the customized route to travel.

In further embodiments, the computer-readable storage medium may include instructions being executable by one or more processors to provide, by the mobile device, real-time information to the user regarding the exercise plan including guidance and user feedback.

In exemplary embodiments, computerized route building systems for creating a customized route to travel may be constructed in accordance with the present technology may include a computer system and a computer-readable application. The computer system may have at least one processor which may further include at least one set of memory. The at least one set of memory may include tangible and non-transitory processor-readable media. The at least one processor may be configured to execute computer-readable instructions on its memory, including instructions for operation of the computerized route building system in accordance with the methods described herein.

The computerized route building system for creating a customized route to travel according to exemplary embodiments permits for the execution of a method according to the present disclosure. The method includes the steps of accessing the computerized route to travel system, which may further include downloading the computer application which retains the system, logging into the system via the interne, or retrieving a copy of the system stored on at least one article of hardware. Next, the method may also include a step of selecting at least one of a route from a pre-determined list of routes and a user designed route. Where the user designs their own route, a plurality of options may be selected such as route shape, topography, terrain, surroundings, and landmarks, as non-limiting examples. Then, the method may include a step of selecting a warm-up or cool-down. Afterwards, the method may include a step of reviewing a map of at least one of the designed and selected route. The method may also include a step of entering a goal which may be determined by at least one of an overall time to complete the route and a pace. Next, the method may include a step of selecting audio. The step of selecting audio may include at least one of a downloaded playlist, at least one of a pre-determined playlist and a customizable playlist that matches a desired beat of the audio file with at least one of an actual pace of a user and a goal pace of a user, and streaming an audio file such as an audiobook and podcasts, as a non-limiting examples.

In certain examples, the computerized route to travel building system may communicate between a plurality of devices. In further examples, the computerized route to travel building system may be synchronized between multiple devices of a single user, such as a smart phone and a smart watch, as non-limiting examples. In other examples, the computerized route to travel building system may communicate between user profiles of a plurality of users. The plurality of users may compare their pace to other users that utilize the computerized route to travel building system.

Once the application for the computerized route to travel building system is downloaded to a device of a user, the route may still be accessible, even where the device loses service or connectivity to satellites. Advantageously, the computerized route to travel building system may be used without the limitations of requiring connectivity to satellites or the usage of a user's data plan with their mobile device provider, as non-limiting examples. Desirably, the computerized route to travel building system may continuously provide the user with saved details about route, the terrain, and the weather.

In other embodiments, the computerized route to travel building system may include an artificial intelligence feature configured to provide recommendations based on the performance of the user. For example, if the user dramatically slowed their pace during a section of the route with more hills, the computerized route to travel building system may recommend more hill training workouts. In another example, if the user takes long breaks between training sessions due to soreness, the computerized route to travel building system may recommend at least one of an ice bath and particular stretches. In a more specific example, the artificial intelligence feature may monitor at least one of an external temperature and a temperature of a user delivered from a device of the user. The artificial intelligence feature may further deliver recommendations to the user to slow down where the least one of the external temperature and the temperature of a user is undesirable for continuing the route at a current pace. In another specific example, the artificial intelligence feature may track the total distance a user has used certain articles of equipment, such as at least one of the footwear and bicycle tires of the user, as non-limiting examples. The artificial intelligence feature may further recommend an appropriate opportunity to replace the article of equipment, such as based on the estimated insufficient tread on at least one of the shoes and the tires of the user. One skilled in the art may select other suitable methods of providing recommendations to users based on their selections, history, and performance, within the scope of the present disclosure.

In some embodiments, the artificial intelligence feature may monitor the location of the user from a Global Positioning System (GPS) feature on the device of the user. In a specific example, the location of the device of the user may be shared with people selected by the user, such as family or close friends, as non-limiting examples. In a more specific example, the artificial intelligence feature may monitor where the user has stopped for longer than a first predetermined period of time and longer than a second predetermined period of time during the route. Where the user has stopped for longer than the first predetermined period of time, an alert to call emergency services is delivered to the device of the user. Where the user has stopped for longer than the second predetermined period of time without dismissing the alert to call emergency services, the computerized route to travel building system may automatically call nearby emergency services and share the location of the device of the user with the emergency services. Advantageously, the location monitoring feature may provide a user with more confidence and peace of mind.

In particular examples, the artificial intelligence feature may be configured to provide real time recommendations to a user during the route. In a more particular example, the real time recommendations may be delivered through at least one of notifications on the device of the user and audio instructions through the device of the user. In an even more particular example, the real time recommendations may include at least one of motivational expressions, notifications such as changes in weather, pollen/air quality levels, traffic, estimated water loss, and personal coaching based on at least one of a goal time of the user, the performance of another selected user, and the computer generated “ghost partner” for personal competition based on the specific goals of the user using current fitness level and stored user data. A skilled artisan may select other suitable methods for delivering real time recommendations to a user, within the scope of the present disclosure.

In other examples, the computerized route to travel building system may be adaptable during the route. In a more specific example, the route data may be adjusted in real time. For instance, the warm-up may be reduced, and the main route may be extended, as a non-limiting example. Additionally, the route may be abandoned and the quickest route to a pre-determined location may be available, as a non-limiting example. In specific examples, the computerized route building system may display and optionally provide directions to common points of interest such as nearest bathrooms, rest areas, and police stations, as non-limiting examples. Advantageously, the adaptable route feature may provide a user with more options to customize their route in real time. Desirably, the adaptable route feature may provide a user with more confidence to complete their route where they may need to shorten their route or find the quickest route to a desired location in real time.

In additional embodiments, users of the computerized route to travel building system may also share their routes and their performances to a community feature within the computerized route to travel building system. The community feature may be configured to allow users to share routes, challenges, workouts, articles/stories, digital images, tips, and recipes, as non-limiting examples. Advantageously, the community feature may increase the motivation and consistency of a user to continue utilizing training programs and more routes.

In particular examples, the device of the user may include at least one of a smartphone, tablet, smart watch, laptop, and other devices that may connect to the internet. The computerized route to travel building system may be accessed on the device of the user through at least one of a WIFI connection, a cellular satellite internet connection, and a hardline internet connection. The user may utilize the computerized route to travel building system through a plurality of menu screens displayed on a screen of the device of the user. A user of the computerized route to travel building system may connect with another user of the computerized route to travel building system where the devices of the users are connected to at least one of a WIFI connection, a cellular satellite internet connection, and a hardline internet connection. Each user of the computerized route to travel building system may simultaneously connect with a plurality of other users of the computerized route to travel building system.

Advantageously, the system is easily accessible and provides desired information regarding the use of the generated routes to a user in one location.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a schematic illustration of a system configured for creating a customized route to travel according to one embodiment of the present disclosure;

FIG. 2 is a flowchart showing a method for creating a customized route to travel according to one embodiment of the present disclosure;

FIG. 3 is another flowchart showing a method for creating a customized route to travel according to one embodiment of the present disclosure;

FIG. 4 is a detailed flowchart showing the step of permitting the user to select the user-determined criteria from FIG. 2;

FIG. 5 is screenshot showing a customized route to travel according to one embodiment of the present disclosure;

FIG. 6 is another screenshot showing a customized route to travel according to one embodiment of the present disclosure; and

FIG. 7 is yet another screenshot showing a customized route to travel according to one embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps may be different in various embodiments, including where certain steps may be simultaneously performed, unless expressly stated otherwise.

“A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” may encompass both an orientation of above and below. The device may be otherwise oriented (rotated 90 degrees or at other orientations) and the spatially relative descriptors used herein interpreted accordingly.

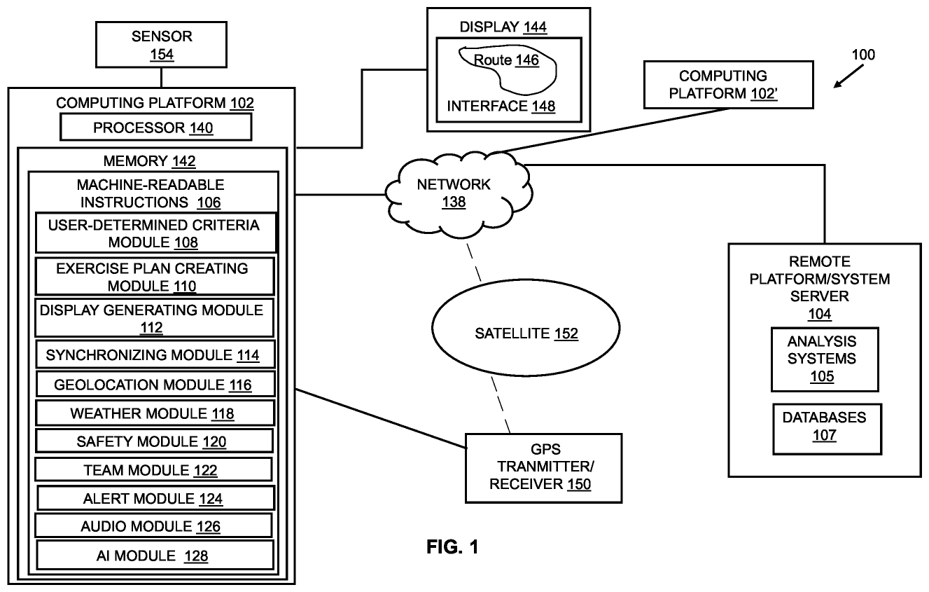

FIG. 1 illustrates a system 100 configured for creating a customized route to travel, for example, as part of a customized exercise plan, in accordance with one or more embodiments.

The system 100 may include one or more computing platforms 102. The computing platforms 102 may be provided in the form of one or more mobile devices such as smartphones, tablet computers, or the like. The one or more computing platforms 102 may be communicably coupled with one or more remote platforms/system server 104. In some cases, users may access the system 100 via remote platforms/system server(s) 104. The one or more computing platforms 102 has a processor 140 and a memory 142, as described in further detail hereinbelow.

The one or more computing platforms 102 may be configured by machine-readable instructions 106 that are stored on the memory 142 of the one or more computing platforms 102. Machine-readable instructions 106 may include modules. The modules may be implemented as one or more of functional logic, hardware logic, electronic circuitry, software modules, and the like. As shown in FIG. 1, the modules may include one or more of user-determined criteria module 108, exercise plan creating module 110, display generating module 112, a synchronizing module 114, a geolocation module 116, a weather module 118, safety module 120, a team module 122, an alert module 124, an audio module 126, artificial intelligence module 128, and/or other modules. It should be appreciated that one skilled in the art may select other suitable modules for use with the one or more computing platforms 102 within the scope of the present disclosure. Additionally, individual modules can be selectively turned on or off by the user.

The computing platform 102 may be configured to include a security module for limiting access to the computing platform 102. The security module may include features to limit unauthorized access to the computing platform 102. The features of the security module may include any features known to those of skill in the art for limiting access to a computing platform 102. Non-limiting examples include, but are not limited to, face recognition, fingerprint sensors, security code, two-step authentication, biometric security features, and other security features known to those of skill in the art.

The computing platform 102, after access has been granted, may be configured to implement one of several modules for capturing information for use in generating the customized route to travel. The modules may all be included or select modules may be implemented, as desired.

The user-determined criteria module 108 may include a variety of exercise related criteria, as a non-limiting example. These criteria may include, but are not limited to, type of exercise, amount of customization, duration of exercise, a pattern of the exercise, a level of difficulty of the exercise, an inclusion of obstacles in the exercise, a general location of the exercise, and a presence of visual stimuli during exercise. The user-determined criteria module 108 may be configured to transmit the user-determined criteria from the mobile device to the system server 104.

More specifically, the user-determined criteria module 108 allows the user to select the type of exercise to be completed by the user. Non-limiting examples of such types of exercise include, but are not limited to, walking, running, cycling, swimming, hiking, and combinations of these exercises.

The user-determined criteria module 108 may further be configured to allow the user to select a duration of the exercise. The duration may be chosen from a length of time, a distance, calories burned, or other similar defining factor.

The user-determined criteria module 108 may be configured to allow the user to select the pattern of the customized route to travel. Non-limiting examples of such patterns include, but are not limited to, a loop, box, out-and-back, and a random selection.

The user-determined criteria module 108 may be configured to allow the user to select the topography for the customized route to travel. Non-limiting examples of such topography include, but are not limited to, small hills, medium hills, large hills, increasing incline, flat surface, and combinations thereof.

The user-determined criteria module 108 may be configured to allow the user to select the surface on which they may be performing the customized route to travel. Non-limiting examples of such surface styles include, but are not limited to, gravel, paved roads, sidewalks, grass, track, and combinations thereof.

The user-determined criteria module 108 may be configured to allow the user to select the environment in which the user may perform the customized route to travel. Non-limited examples include, but are not limited to, in a forest, in a park, in a city, in a town, off-road, and a random selection.

The user-determined criteria module 108 may be configured to allow the user to select the visual stimuli that the user may see while performing the customized route to travel. Non-limited examples include, but are not limited to, landmarks, bodies of water, famous sites, and other similar visual stimuli.

The user-determined criteria module 108 may be configured to allow the user to add a warmup, cooldown, or both a warmup and cooldown to the customized route to travel.

The exercise plan creating module 110 may be configured to create, by the system server 104, the customized route to travel based on user-determined criteria. The exercise plan creating module 110 may be configured to transmit the customized route to travel from the system server 104 to the computing platform 102 such as the mobile device through the wide area network or other similar transmissions.

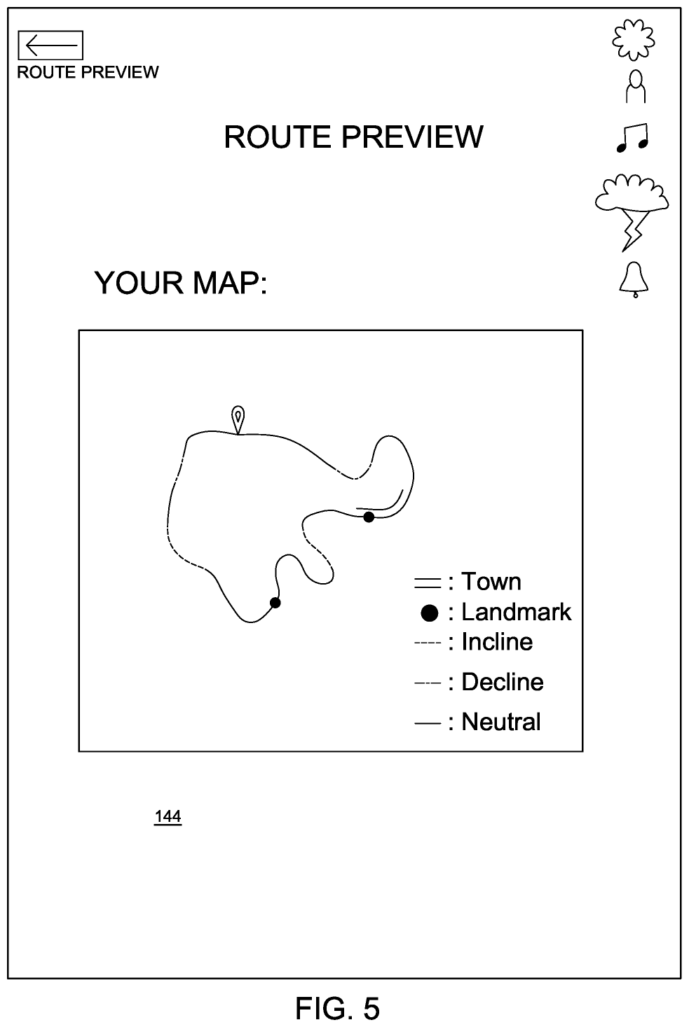

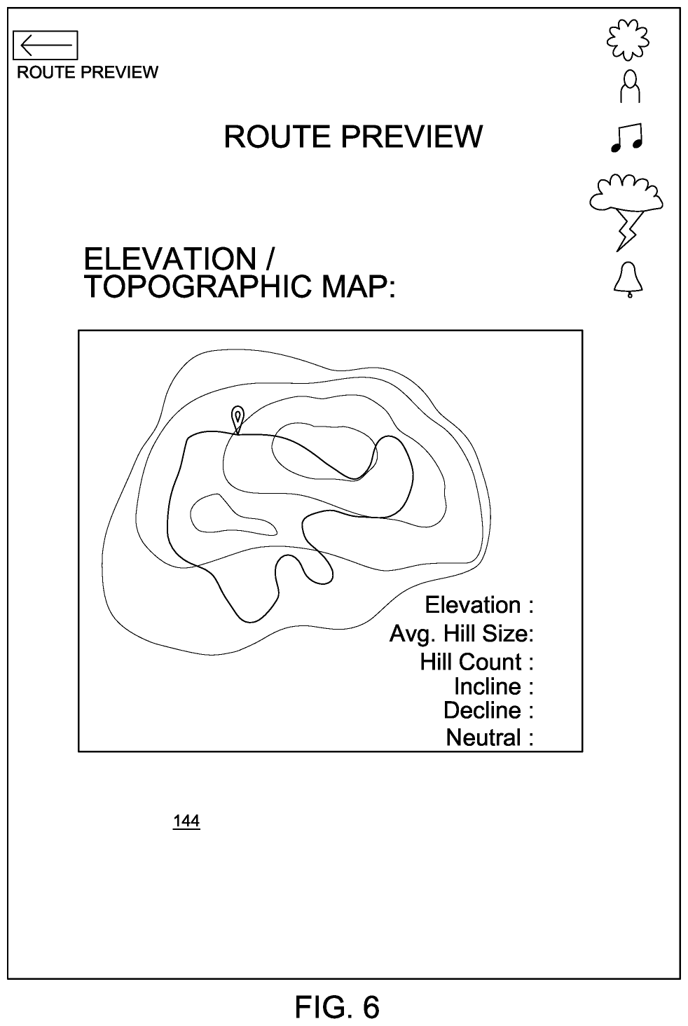

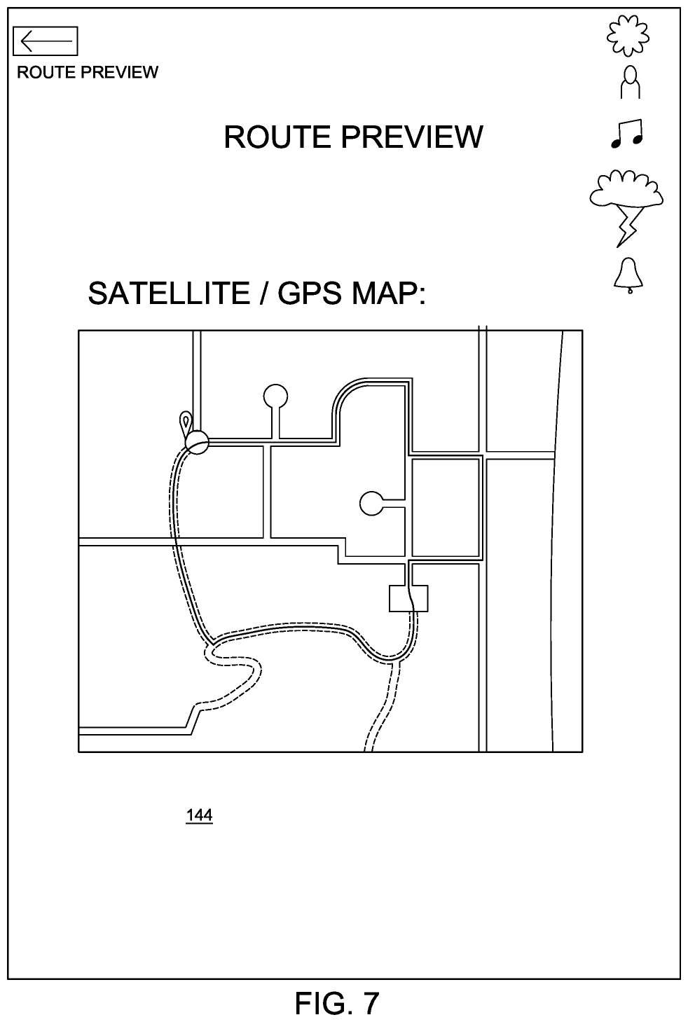

The display generating module 112 may be configured to generate a display 144 of the customized route to travel on the mobile display of the computing platform 102 such as the mobile device. The display may provide more than one route option, and may allow the user to select from several route options. The display 144 of the customized route to travel may include a visual map 146 of a route associated with the customized route to travel. The display 144 may be 2-dimensional, a 3-dimensional or a rotatable 3-dimensional display of the customized route to travel that may be viewed on the mobile display of the computing platform 102, using systems known to those of skill in the art. The display generating module 112 may also provide elevational and topological detail of the customized route to travel on the mobile display of the computing platform 102. The display generating module 112 may also provide detailed information regarding the customized route to travel, examples of which are shown in FIGS. 5-7. This detailed information may include, but is not limited to, percent (%) incline, percent (%) of a specific surface (e.g., grass, paved road, gravel), weather conditions, and other similar detailed information. The plan may be downloaded, transferred using wireless technology, or uploaded to a shared device.

In other examples, the display generating module 112 may also enable the user, by the human interface 148 of the computing platform 102, to alter the exercise plan during use of the customized route to travel and may provide real-time information to the user regarding the exercise plan including guidance and user feedback. In a more specific example, the customized exercise plan data may be adjusted in real time. For instance, additional feature may be added, such as a warm-up and the duration may be extended, as a non-limiting example. Additionally, the customized route to travel may be cancelled and the quickest route to a pre-determined location (such as the start of the route) may be available, as a non-limiting example. Advantageously, the adaptable route feature may provide a user with more options to customize their customized route to travel in real time or to adjust the route after completion. Desirably, the adaptable route feature may provide a user with more confidence to complete their route where they may need to shorten their route or find the quickest route to a desired location in real time.

In some cases, the system 100 may further include a synchronizing module 114 configured to automatically synchronize all information related to the customized route to travel to the system server 104. The data can be stored on a specialized server or using cloud storage. The synchronizing module 114 may include synchronizing the customized route to travel created, performance data, times, weather conditions, music and any other material included as part of a customized route to travel. The route data that may be both stored and synchronized may include the following non-limiting examples: total distance of rout, average pace, pace per mile/kilometer, average MPH/KPH, MPH/KPH per each mile/kilometer, start time, end time, duration of route, time of each mile/kilometer, average heart rate, heart rate of each mile/kilometer, temperature during route, weather during route, total percent of incline/decline, percent of incline/decline of each mile/kilometer, average route elevation, elevation of each mile/kilometer, set goal, whether the goal was met/not met/surpassed, route conditions, overall terrain, terrain of each mile/kilometer, total calories burned, calories burned of each mile/kilometer, performance rating (poor, average, good), route level of difficulty, performance of each mile/kilometer (poor, struggled, excelled, increased, slowed down), overall performance rating. This information may be stored as raw data or in graphical representation. The graphical representations may include comparisons to prior activities. Additionally, the synchronizing module 114 can compare post route data.

In some cases, the system 100 may further include at least one geolocation module 116, such as a global positioning system (GPS), in communication with at least one of the computing platform 102 and the system server 104, and the step of transmitting the customized route to travel includes determining a geolocation of the computing platform 102 such as the mobile device within the customized route to travel. Thus, the computing platform 102, such as the mobile device, may include a transmitter/receiver 150 enabling communication with a geolocation system, such as a GPS, that may communicate via a satellite 152.

The system 100 may further include a weather module 118. The weather module 118 may provide the forecast for the location selected by the user-determined criteria. Additionally, the weather module 118 may provide clothing recommendations, possible routes conditions (e.g., icy, muddy, etc.), traffic updates, and pollen and air quality reports.

The system 100 may further include at least one sensor 154 in communication with the computing platform 102 such as the mobile device. The sensor 154 may be configured to assess and communicate to the system server 104 one or more user attributes selected from the group consisting of body temperature, hydration, and pulse. Non-limiting examples of such attributes include, but are not limited to, body temperature, hydration, blood pressure, and pulse. The user attributes information may be relayed to the system and the attribute may be quantified. This assessment may be compared against a predetermined acceptable range, such that if the assessed user attribute quantification falls outside of the predetermined acceptable range an alert may be provided to the user on the mobile display of the mobile device. Nonlimiting examples of the sensors include, but are not limited to, accelerometers, gyroscopes, electromagnetic sensors, biological sensors, and optical tracking sensors. It should further be appreciated that a skilled artisan may employ different types and numbers of the sensors 154 as required by the procedure or situation within which the system 100 is being used.

The sensors may also be used to monitor user movement. The sensors may be configured to detect prolonged stops or breaks during a customized route to travel. Detection of a prolonged stop triggers an alert to the user on the mobile display of the computing platform 102 such as the mobile device. The user must then provide an interaction with the computing platform 102 within a predetermined amount of time, such that absent such an interaction an emergency alert may be sent by the computing platform 102 to emergency personnel that are in close proximity to the user based on geolocation information.

In a specific example, the computing platform 102 location may be shared via the network with one or more other computing platforms 102′ on mobile devices of third party users also running the system 100, such as the family or friends of the user', such as family or close friends, as non-limiting examples. In a more specific example, the system 100 may monitor where the user has stopped for longer than a first predetermined period of time during the customized route to travel, such as if the user stops for over three (3) minutes. Where the user has stopped for longer than the first predetermined period of time, an alert to call emergency services is delivered to the computing platform 102 of the user. Where the user takes longer than the second predetermined period of time, such as 20 seconds, without dismissing the alert to call emergency services, the system 100 may automatically call nearby emergency services, share the location of the user with the emergency services, and alert other devices that are synchronized with the user. Advantageously, the location monitoring feature may provide a user with more confidence and peace of mind.

The system 100 may further include the safety module 120 configured that may be initiated by the user during performance of the customized route to travel. In some cases, the safety module 120 may be configured to activate a tracking system for determining a user proximity to police, fire department, and bathrooms. The safety module 120 can also include the ability to avoid predetermined areas.

The system 100 may further include a team module 122 configured to allow groups of people to share exercise plan information, compare training routines, and compete with each other. The team module 122 may be configured to allow users to sign up for a group and share information using another computing platform 102′ on another mobile device, such that each individual using the team module 122 can be seen on a shared map with personal markers indicating each individual. Non-limiting examples of such information include, but are not limited to, synchronized training/exercise information, music, compare training routines, create competitions, share recipes, share pictures, and create a scrapbook. Additionally, the data may include the following non-limiting examples, which can be compared between other team module users: total distance of route, average pace, pace per mile/kilometer, average MPH/KPH, MPH/KPH per each mile/kilometer, start time, end time, duration of route, time of each mile/kilometer, average heart rate, heart rate of each mile/kilometer, temperature during route, weather during route, total percent of incline/decline, percent of incline/decline of each mile/kilometer, average route elevation, elevation of each mile/kilometer, set goal, whether the goal was met/not met/surpassed, route conditions, overall terrain, terrain of each mile/kilometer, total calories burned, calories burned of each mile/kilometer, performance rating (poor, average, good), route level of difficulty, performance of each mile/kilometer (poor, struggled, excelled, increased, slowed down), overall performance rating. This information may be stored as raw data or in graphical representation. The graphical representations may include comparisons to prior activities. The team module 122 may also be configured to provide information about nearby clubs or events related to the exercise.

In certain examples, the system 100 may communicate between a plurality of devices using a community module. In further examples, the system 100 may be synchronized, using the synchronizing module 114, between multiple devices of a single user, such as a smart phone and a smart watch, as non-limiting examples. In other examples, the system 100 may communicate between user profiles of a plurality of users. For example, a plurality of users may compare their pace to other users that utilize the system. Advantageously, the community feature may increase the motivation and consistency of a user to continue utilizing training programs and more routes. The community module may also allow you to invite friends to join the community module.

The system 100 may further include an alert module 124 configured to assess the customized exercise training plan, assess the performance of the customized training plan, and provide alerts for potential training problems such as overtraining and tire or shoe tread wear. The alert module 124 may provide warnings to the user about such training problems and offer training suggestions, physical therapists, local stores with equipment, when the user has allowed for sufficient recovery time between usages, and other similar recommendations. One skilled in the art may select other suitable recommendations to users based on their selections, history, and performance, within the scope of the present disclosure.

In some cases, the system 100 may further include an audio module 126 configured to enable audio that may be integrated into the customized route to travel. Non-limiting examples of such audio include, but are not limited to include music, podcasts, books, and playlists. The audio module 126 may also include smart beat technology that works within the customized route to travel. The smart beat technology may match the pace needed for the user to achieve the goal selected in the user-determined criteria 108, such that if the user is falling off of the required pace the beat will speed up until the required pace is achieved. Alternatively, the smart beat technology may either match your pace or maintain a constant pace. Alternatively, the audio module 126 may include a voice option/audio coach that may provide motivation, instruction, and directions during performance of the customized route to travel. The coach option may include different voice options. The coach option may also include providing the data and analytics to an AI or live coach in real time.

The system 100 may also include an artificial intelligence (AI) or machine learning module 128, using standard machine learning systems know to those of skill in the art, which is configured to receive data regarding performance of the customized route to travel, assess the performance, and provide feedback regarding the performance. For example, the AI module 128 may provide post route recommendations, such as shorten your stride at certain points, lengthen stride at certain points, or increase muscles to increase performance. Additionally, the AI module 128 may adjust the customized route to travel for future uses based on past performance. For example, if the user struggled with hills, the AI module 128 may offer route suggestions to improve performances in those locations where the user struggled, or the AI module 128 may create a training plan to increase the muscles needed for better performance in the locations where the user struggled.

In other embodiments, the AI module 128 may be configured to analyze data compiled from the exercise plan and may modifying future customized route to travels from the analysis of the user data collected during the customized route to travel, the modifications being selected from route recommendations, including a warmup, including a cool-down, and altering route difficulties, the step of analyzing data further includes recommending technical exercise improvements selected from the group consisting of altering customized route to travel routes, post route workout advice, and recovery techniques and the analyzing step further includes generating post workout graphs and charts. The data that may be analyzed may include the following non-limiting examples: total distance of rout, average pace, pace per mile/kilometer, average MPH/KPH, MPH/KPH per each mile/kilometer, start time, end time, duration of route, time of each mile/kilometer, average heart rate, heart rate of each mile/kilometer, temperature during route, weather during route, total percent of incline/decline, percent of incline/decline of each mile/kilometer, average route elevation, elevation of each mile/kilometer, set goal, whether the goal was met/not met/surpassed, route conditions, overall terrain, terrain of each mile/kilometer, total calories burned, calories burned of each mile/kilometer, performance rating (poor, average, good), route level of difficulty, performance of each mile/kilometer (poor, struggled, excelled, increased, slowed down), overall performance rating. This may also be include mapping the performance of the user on the same route at different points in time. This may include collecting data with regard to specific user performance. In other words, the system 100 can select a portion of a route and collect performance data regarding that section each time the user completes that portion of the route. The AI module 128 may provide user feedback based on the data. This information may be stored as raw data or in graphical representation. The graphical representations may include comparisons to prior activities.

With respect to the warmup and cooldown, it should be appreciated that the user may add the warmup or the cooldown to the main route. If the user adds this to the main route, the user may set up a desired distance. When that desired distance is reached the data may be saved separately from the main route. If the user ends a warmup or cooldown early, or if the user goes longer, the user can go into the system 100 after the route is uploaded and correct the warmup or cooldown distances. The system 100 may then automatically readjust the data and statistics.

Alternatively, the user may select to keep the warmup or cooldown separate from the main route. In this case, the user can add specifications, but the warmup or cooldown will not be attached to the main route or start and finish in a location within the system away from the main route. The data collected will not be incorporated or affect the data collected from the main route. The data sets will remain separate within the system 100 in this case.

It should be understood that the post-workout graphs may be visual graphics on the mobile display to depict (e.g., using color coding) for the user the areas in need of additional strengthening. The graphics may include a variety of graph analytics. The graph analytics may include color coding to show areas of difficulty. The AI module 128 may also be configured to create a ghost chase to provide the user with a preview of potential performance on a customized route to travel. For example, the user may input their fastest time into the module. This then may allow the user to compete against their fastest time on a specific route. The ghost chase may be based on previously collected data on previous routes or just input in as a time (e.g., the time can be broken down specifically per mile or kilometer, i.e. 6 min. first mile, 5 min. second mile).

In other embodiments, the AI module 128 may be configured to provide recommendations based on the performance of the user. In other words, the AI module 128 uses completed workouts to create more detailed and accurate routs and workouts for future uses. For example, if the user dramatically slowed their pace during a section of the route with more hills, the AI module 128 may recommend more hill training workouts. In another example, if the user takes long breaks between training sessions due to soreness, the AI module 128 may recommend at least one of an ice bath and particular stretches. In a more specific example, the AI module 128 may monitor, in connection with the sensors, at least one of an external temperature and a temperature of a user delivered from a device of the user. The AI module 128 may further deliver recommendations to the user to slow down where the least one of the external temperature and the temperature of a user is undesirable for continuing the customized route to travel at a current pace. In another specific example, the AI module 128 may track the total distance a user has used certain articles of equipment, such as at least one of the footwear and bicycle tires of the user, as non-limiting examples. Other non-limiting examples of recommendations include, but are not limited to, more routes with hills, strengthen specific muscles, longer strides, shorter strides, increase lung capacity and methods of doing so such as breathing exercises, altitude training, specific workouts, eat more calories, food and beverage recommendations, more water consumption, use of ice baths, stretching exercises, more incline training, less inclines, terrain recommendations, recommend goals, places where user struggled or excelled, recommendations for timing of breaks.

In particular examples, the AI module 128 may be configured to provide real time recommendations to a user during the customized route to travel. In a more particular example, the real time recommendations may be delivered through at least one of notifications on the mobile display of the mobile device of the user and audio instructions through the device of the user. In an even more particular example, the real time recommendations may include at least one of motivational expressions, notifications such as changes in weather, pollen/air quality levels, traffic, estimated water loss, and personal coaching based on at least one of a goal time of the user, the performance of another selected user, and the computer generated “ghost partner” for personal competition based on the specific goals of the user. A skilled artisan may select other suitable methods for delivering real time recommendations to a user, within the scope of the present disclosure.

Additionally, once the customized route to travel is downloaded, transferred, or uploaded to a device of a user, the customized route to travel may still be accessible, even where the device loses service or connectivity to satellites. If there is no connectivity the system 100 can provide a pedometer, timer and compass to aid the user in completing the route. Advantageously, the system 100 may be used without the limitations of requiring connectivity to satellites or the usage of a user's data plan with their mobile device provider, as non-limiting examples. Desirably, the system 100 may continuously provide the user with saved details about route, the terrain, and the weather.

In some cases, the one or more computing platforms 102, may be communicatively coupled to the remote platforms/system server(s) 104. In some cases, the communicative coupling may include communicative coupling through a networked environment 138. The networked environment 138 may be a radio access network, such as LTE or 5G, a local area network (LAN), a wide area network (WAN) such as the Internet, or wireless LAN (WLAN), for example. It will be appreciated that this is not intended to be limiting, and that the scope of this disclosure includes implementations in which one or more computing platforms 102 and remote platforms/system server(s) 104 may be operatively linked via some other communication coupling. The one or more one or more computing platforms 102 may be configured to communicate with the networked environment 138 via wireless or wired connections. In addition, in an embodiment, the one or more computing platforms 102 may be configured to communicate directly with each other via wireless or wired connections. Examples of one or more computing platforms 102 may include, but is not limited to, smartphones, wearable devices, tablets, laptop computers, desktop computers, Internet of Things (IoT) device, or other mobile or stationary devices. In an embodiment, system 100 may also include one or more hosts or servers, such as the one or more remote platforms/system servers 104 connected to the networked environment 138 through wireless or wired connections. According to one embodiment, remote platforms/system servers 104 may be implemented in or function as base stations (which may also be referred to as Node Bs or evolved Node Bs (eNBs)). In other embodiments, remote platforms/system servers 104 may include web servers, mail servers, application servers, etc. According to certain embodiments, remote platforms/system servers 104 may be standalone servers, networked servers, or an array of servers.

In one embodiment the computing platform 102 may be a mobile device. The mobile device may have a human interface for inputting user-determined criteria into the module user-determined module 108. The mobile device can be a phone, a smart watch, or any other mobile devices known to those of skill in the art. The mobile device may include a mobile display for displaying the customized route to travel compiled by the system server 104. The display may be a liquid crystal display or other display known to those of skill in the art to be used in connection with the method and system of the present disclosure. The display may also include controllers for use by the user in inputting user-determined criteria into the system 100. Non-limiting examples of such controllers include, but are not limited to, a touch pad/screen, a switch, voice commands mechanisms, and other user interface mechanisms.

The one or more computing platforms 102 may include one or more processors 140 for processing information and executing instructions or operations. One or more processors 140 may be any type of general or specific purpose processor. In some cases, multiple processors 140 may be utilized according to other embodiments. In fact, the one or more processors 140 may include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. In some cases, the one or more processors 140 may be remote from the one or more computing platforms 102, such as disposed within a remote platforms/system server 104 like the one or more remote platforms/system servers 104 of FIG. 1.

The one or more processors 140 may perform functions associated with the operation of system 100 which may include, for example, precoding of antenna gain/phase parameters, encoding and decoding of individual bits forming a communication message, formatting of information, and overall control of the one or more computing platforms 102, including processes related to management of communication resources.

The one or more computing platforms 102 may further include or be coupled to a memory 142 (internal or external), which may be coupled to one or more processors 140, for storing information and instructions that may be executed by one or more processors 140. Memory 142 may be one or more memories and of any type suitable to the local application environment and may be implemented using any suitable volatile or nonvolatile data storage technology such as a semiconductor-based memory device, a magnetic memory device and system, an optical memory device and system, fixed memory, and removable memory. For example, memory 142 may consist of any combination of random-access memory (RAM), read-only memory (ROM), static storage such as a magnetic or optical disk, hard disk drive (HDD), or any other type of non-transitory machine or computer readable media. The instructions stored in memory 142 may include program instructions or computer program code that, when executed by one or more processors 140, enable the one or more computing platforms 102 to perform tasks as described herein.

In some embodiments, one or more computing platforms 102 may also include or be coupled to one or more antennas for transmitting and receiving signals and/or data to and from one or more computing platforms 102. The one or more antennas may be configured to communicate via, for example, a plurality of radio interfaces that may be coupled to the one or more antennas. The radio interfaces may correspond to a plurality of radio access technologies including one or more of LTE, 5G, WLAN, Bluetooth, near field communication (NFC), radio frequency identifier (RFID), ultrawideband (UWB), and the like. The radio interface may include components, such as filters, converters (for example, digital-to-analog converters and the like), mappers, a Fast Fourier Transform (FFT) module, and the like, to generate symbols for a transmission via one or more downlinks and to receive symbols (for example, via an uplink).

The remote platforms/system server 104 may include analysis systems 105 to analyze the user-determined criteria, and other inputs by the user and in communication with the computing platform 102. The remote platforms/system server 104 may include databases of information 107. The information may include any of the information needed to be accessed by the system 100. The databases 107 may be used, in conjunction with the user-determined criteria, to generate or create the customized route to travel. The analysis system 105 contained within the remote platforms/system server 104 may jointly analyze the information stored within the databases 107 and the user-determined criteria to transform the data and user-determined criteria into an executable customized route to travel. This analysis creates a customized route to travel that may be transmitted to the user on the mobile display, examples of which are shown in FIGS. 5-7.

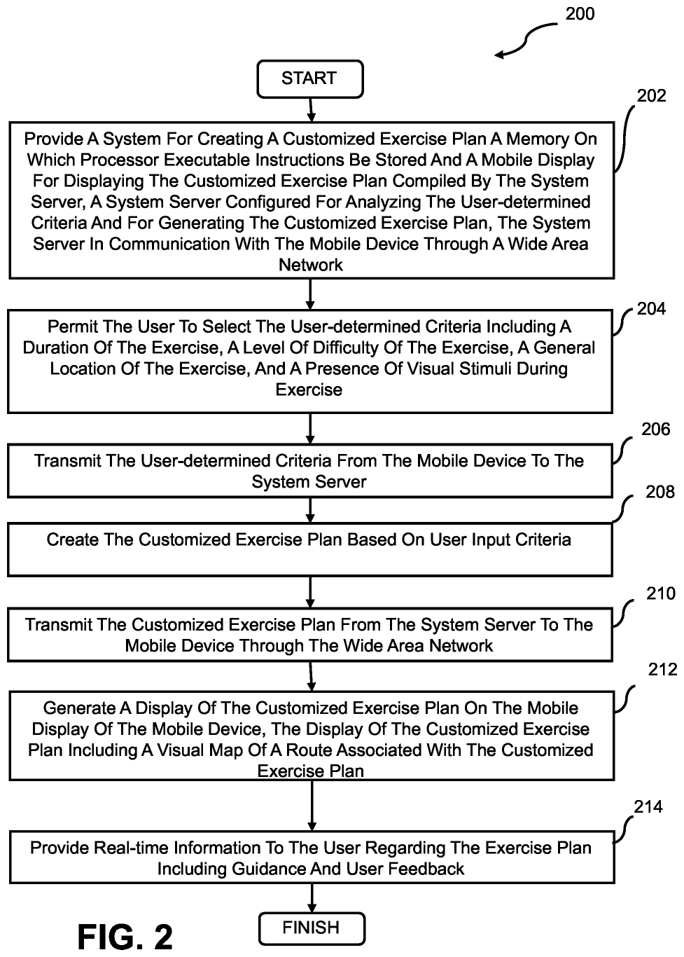

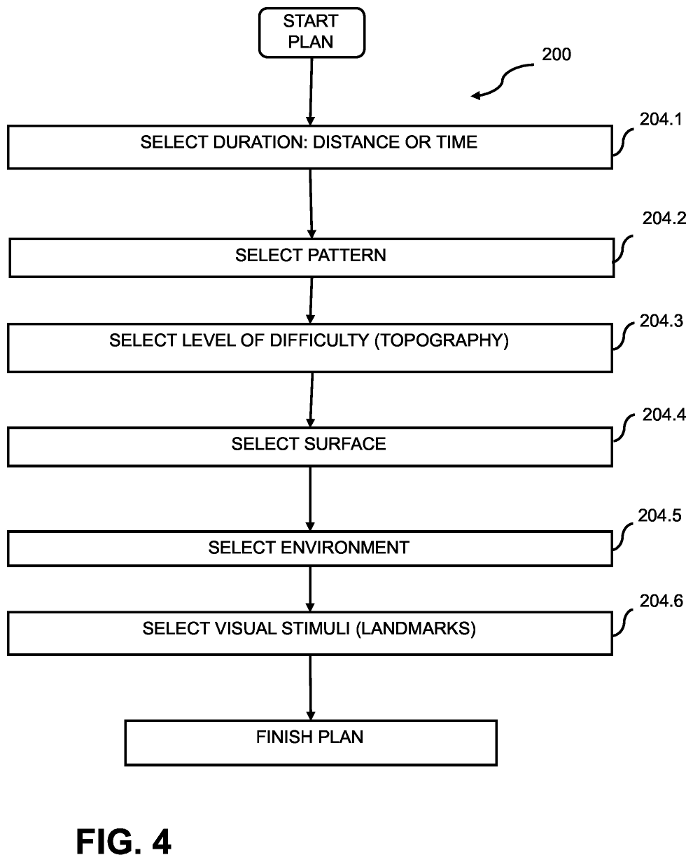

FIGS. 2 and 4 illustrates an example flow diagram of a method 200, according to one embodiment. The method 200 may include providing a system for creating a customized route to travel, the system including a mobile device having a human interface for inputting user-determined criteria, a memory on which processor executable instructions being stored, a processor in communication with the human interface and the memory, and a mobile display for displaying the customized route to travel compiled by the system server, a system server configured for analyzing the user-determined criteria and for generating the customized route to travel, the system server in communication with the mobile device through a wide area network at block 202. The method 200 may include permitting the user, by human interface of the mobile device, to select the user-determined criteria including a duration of the exercise at block 204.1, a pattern of the exercise at block 204.2, a level of difficulty (i.e. the topography) of the exercise at block 204.3, an inclusion of obstacles/selection of surface of the exercise at block 204.4, a general location or environment of the exercise at block 204.5, and a presence of visual stimuli (i.e. landmarks) during exercise at block 204.6. The method 200 may include transmitting the user-determined criteria from the mobile device to the system server at block 206. The method 200 may include creating, by the system server, the customized route to travel based on user-determined criteria at block 208. The method 200 may include transmitting the customized route to travel from the system server to the mobile device through the wide area network at block 210. The method 200 may include generating a display of the customized route to travel on the mobile display of the mobile device, the display of the customized route to travel including a visual map of a route associated with the customized route to travel at block 212. The method 200 may include providing, by the mobile device, real-time information to the user regarding the exercise plan including guidance and user feedback at block 214.

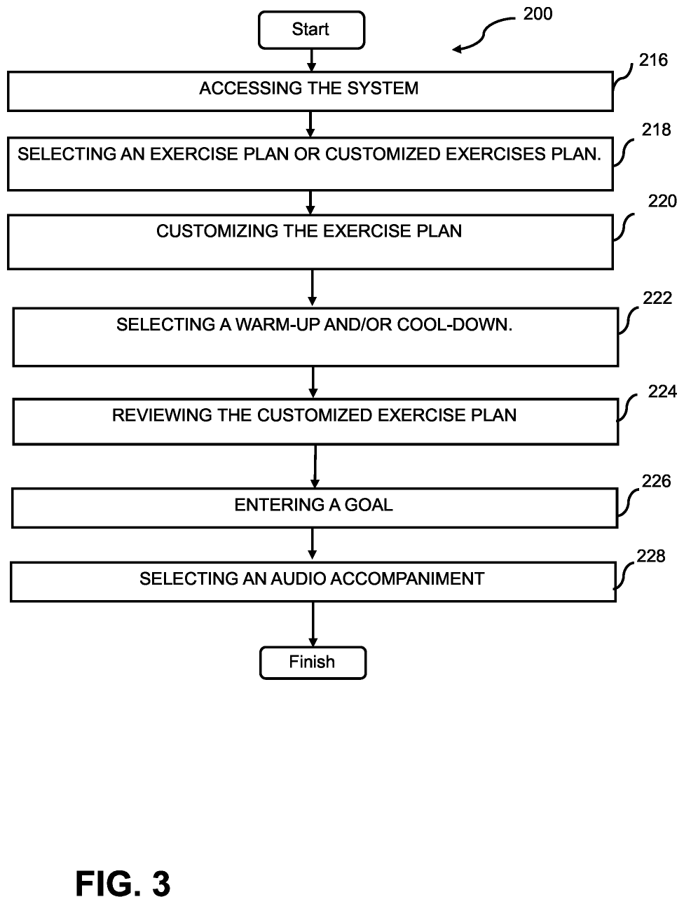

In particular embodiments, as shown in FIG. 3 the system may be configured to perform various operations that may be defined as method steps related to building one or more customized route to travels. Such methods may include the steps of accessing the system at block 216, which may further include downloading a computer application which retains the system, logging into the system via the interne, or retrieving a copy of the system stored on at least one article of hardware. Next, the method may also include a step of selecting at least one of an exercise plan from a pre-determined list of exercise plans and a customized route to travel at block 218. Where the user designs their own customized route to travel at block 220, a plurality of options may be selected such as route shape, topography, terrain, surroundings, and landmarks, as non-limiting examples. Then, the method may include a step of selecting a warm-up or cool-down at block 222. Afterwards, the method may include a step of reviewing a map of at least one of the designed and selected exercise plan at block 224. The method may also include a step of entering a goal which may be determined by at least one of an overall time to complete the route and a pace at block 226. Next, the method may include a step of selecting audio at block 228. The step of selecting audio may include at least one of a downloaded playlist, a pre-determined playlist that matches the beat with a pace of a user, and streaming an audio file such as an audiobook, as a non-limiting examples.

In a certain example, the method may include communicating between a plurality of devices. In specific examples, the system may be synchronized between multiple devices of a single user, such as a smart phone and a smart watch, as non-limiting examples. In other examples, the method may include communicating between user profiles of a plurality of users, wherein the users may compare their pace to other users that utilize the system for creating a customized route to travel.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods may be made within the scope of the present technology, with substantially similar results.

What is claimed is:

1. A method, of creating a customized route to travel, comprising the steps of:
providing a system for creating a customized route to travel, the system including:
a mobile device having a human interface for inputting user-determined criteria, a memory on which processor executable instructions being stored, a processor in communication with the human interface and the memory, and a mobile display for displaying the customized route to travel compiled by the system server, a system server configured for analyzing the user-determined criteria and for generating the customized route to travel, the system server in communication with the mobile device through a wide area network;
permitting the user, by human interface of the mobile device, to select the user-determined criteria including a duration of the exercise, a pattern of the exercise, a level of difficulty of the exercise, an inclusion of obstacles in the exercise, a general location of the exercise, and a presence of visual stimuli during exercise;
transmitting the user-determined criteria from the mobile device to the system server;
creating, by the system server, the customized route to travel based on user-determined criteria;
transmitting the customized route to travel from the system server to the mobile device through the wide area network;
generating a display of the customized route to travel on the mobile display of the mobile device, the display of the customized route to travel including a visual map of a route associated with the customized route to travel; and
providing, by the mobile device, real-time information to the user regarding the exercise plan including guidance and user feedback.

2. The method of claim 1, wherein the generating step further includes generating the display as a rotatable 3D display of the customized route to travel on the mobile display of the mobile device.

3. The method of claim 2, wherein the generating step further includes providing elevational and topological detail of the customized route to travel on the mobile display of the mobile device.

4. The method of claim 2, wherein the providing step includes permitting the user, by the human interface of the mobile device, to alter the exercise plan during use of the customized route to travel.

5. The method of claim 1, wherein the system further includes at least one global positioning satellite in communication with at least one of the mobile device and the system server, and the step of transmitting the customized route to travel includes determining a geolocation of the mobile device within the customized route to travel.

6. The method of claim 1, wherein the system further includes at least one sensor in communication with the mobile device, the sensor configured to assess and communicate to the system server one or more user attributes selected from the group consisting of body temperature, hydration, and pulse, and wherein the user attributes outside of a predetermined acceptable range trigger an alert to the user on the mobile display of the mobile device.

7. The method of claim 1, wherein the system further includes an accelerometer in communication with the mobile device, the accelerometer configured to monitor a user movement using the accelerometer.

8. The method of claim 7, further comprising a step of monitoring the user movement to detect a prolonged stop during the customized route to travel, wherein detection of the prolonged stop triggers an alert to the user on the mobile display of the mobile device and, absent an interaction from the user with the mobile device, an emergency alert is sent by the mobile device to emergency personnel.

9. The method of claim 1, further comprising a step of initiating a safety module to run during the customized route to travel.

10. The method of claim 9, wherein the safety module is configured to activate a tracking system for determining a user proximity to police, fire department, and bathrooms.

11. The method of claim 1, further comprising a step of initiating a team module for allowing multiple users to access data of other mobile devices of other users during and after execution of the customized route to travel.

12. The method of claim 11, wherein the step of initiating the team module includes activating a comparative data sharing module for sharing data with the other mobile devices.

13. The method of claim 1, further comprising a step of analyzing data compiled from the exercise plan using an artificial intelligence module.

14. The method of claim 13, wherein the step of analyzing data further include modifying future customized route to travels from the analysis of the user data collected during the customized route to travel, the modifications being selected from route recommendations, including a warmup, including a cool-down, and altering route difficulties.

15. The method of claim 13, wherein the step of analyzing data further includes recommending technical exercise improvements selected from the group consisting of altering customized route to travel routes, post route workout advice, and recovery techniques.

16. The method of claim 1, wherein the step of creating the customized route to travel further includes selecting, by the user with the mobile device, the type of exercise to be performed by the user while using the customized route to travel.

17. A system, comprising:
one or more hardware processors configured by machine-readable instructions to:
provide a system for creating a customized route to travel, the system including a mobile device having a human interface for inputting user-determined criteria, a memory on which processor executable instructions be stored, a processor in communication with the human interface and the memory, and a mobile display for displaying the customized route to travel compiled by the system server, a system server configured for analyzing the user-determined criteria and for generating the customized route to travel, the system server in communication with the mobile device through a wide area network;
permit the user, by human interface of the mobile device, to select the user-determined criteria including a duration of the exercise, a pattern of the exercise, a level of difficulty of the exercise, an inclusion of obstacles in the exercise, a general location of the exercise, and a presence of visual stimuli during exercise;
transmit the user-determined criteria from the mobile device to the system server;
create, by the system server, the customized route to travel based on user-determined criteria;
transmit the customized route to travel from the system server to the mobile device through the wide area network;
generate a display of the customized route to travel on the mobile display of the mobile device, the display of the customized route to travel including a visual map of a route associated with the customized route to travel; and
provide, by the mobile device, real-time information to the user regarding the exercise plan including guidance and user feedback.

18. The system of claim 32, wherein the generating step further includes generating the display as a rotatable 3D display of the customized route to travel on the mobile display of the mobile device.

19. A non-transient computer-readable storage medium comprising instructions being executable by one or more processors to perform a method, the method comprising:
providing a system for creating a customized route to travel, the system including a mobile device having a human interface for inputting user-determined criteria, a memory on which processor executable instructions being stored, a processor in communication with the human interface and the memory, and a mobile display for displaying the customized route to travel compiled by the system server, a system server configured for analyzing the user-determined criteria and for generating the customized route to travel, the system server in communication with the mobile device through a wide area network;
permitting the user, by human interface of the mobile device, to select the user-determined criteria including a duration of the exercise, a pattern of the exercise, a level of difficulty of the exercise, an inclusion of obstacles in the exercise, a general location of the exercise, and a presence of visual stimuli during exercise;
transmitting the user-determined criteria from the mobile device to the system server;
creating, by the system server, the customized route to travel based on user-determined criteria;
transmitting the customized route to travel from the system server to the mobile device through the wide area network;
generating a display of the customized route to travel on the mobile display of the mobile device, the display of the customized route to travel including a visual map of a route associated with the customized route to travel; and
providing, by the mobile device, real-time information to the user regarding the exercise plan including guidance and user feedback.

20. The computer-readable storage medium of claim 19, wherein the generating step further includes generating the display as a rotatable 3D display of the customized route to travel on the mobile display of the mobile device.
